(19) United States
(12) Patent Application Publication
(10) Pub. No.: US 2025/0299809 A1
(43) Pub. Date: Sep. 25, 2025

(54) SIMPLIFIED SERVICE REQUESTS FOR MEDICAL IMAGING SYSTEMS

(71) Applicant: KONINKLIJKE PHILIPS N.V., EINDHOVEN (NL)

(72) Inventor: Steffen WEIß, HAMBURG (DE)

(21) Appl. No.: 19/082,279

(22) Filed: Mar. 18, 2025

Related U.S. Application Data
(60) Provisional application No. 63/568,058, filed on Mar. 21, 2024.

(51) Int. Cl. G16H 40/40; G16H 40/67

(52) U.S. Cl. CPC G16H 40/40; G16H 40/67

(57) ABSTRACT

A medical imaging system (1) includes an electronic controller (10) including a display (24), the electronic controller configured to implement an imaging workflow graphical user interface (GUI) (26) enabling an associated user to set up an imaging workflow, execute the imaging workflow to acquire imaging data of the associated imaging subject using a medical imaging device (2), and display at least one medical image of the associated imaging subject generated by processing of the imaging data acquired by the execution of the imaging workflow; a problem reporting GUI dialog (28) hosted on the electronic controller and integrated with the workflow UI, wherein the problem reporting GUI is configured to receive a problem report indicating a problem with the medical imaging device; and a problem remediation tool (30) configured to perform at least one remedial action in response to receiving the problem report via the problem reporting GUI dialog.

### CROSS REFERENCE TO RELATED APPLICATIONS

This application claims the benefit of U.S. Provisional Patent Application No. 63/568,058 filed Mar. 21, 2024. This application is hereby incorporated by reference herein.

### FIELD

The following relates generally to the medical imaging device operational and maintenance arts, medical device service request creation and management arts, and related arts.

### BACKGROUND

Medical imaging devices are highly computerized. Some examples include medical imaging devices such as magnetic resonance imaging (MRI) scanners, computed tomography (CT) scanners, positron emission tomography (PET) scanners, ultrasound imaging systems, and so forth; image-guided therapy (IGT) systems employing such medical imaging devices; patient monitors, mechanical respirators, and so forth. Medical imaging devices typically include (or viewed alternatively, are operatively connected with) an electronic controller that implements an imaging workflow graphical user interface (GUI) enabling the operator (i.e., user) to set up an imaging workflow, execute the imaging workflow to acquire imaging data of a patient (or other imaging subject) using the medical imaging device, and display medical images of the patient generated by processing of the imaging data acquired by the execution of the imaging workflow. In a typical design, the user can retrieve a parameterized imaging workflow and modify parameter values thereof using the imaging workflow GUI to set up the imaging workflow for the specific patient. Furthermore, the medical imaging device (and more specifically its electronic controller) is typically connected to the hospital electronic data network and often thereby also to the Internet. These wired and/or wireless network connections enable functionality such as uploading medical images to a Picture Archiving and Communication System (PACS), downloading medical imaging device software or firmware updates from the vendor of the medical imaging device, connecting the medical imaging device with a radiology scheduling system, connecting the medical imaging device with an electronic medical or health record (EMR or EHR) database to retrieve relevant patient information, and/or so forth.

Given the large number of components making up a typical medical imaging device (which can number in the hundreds, thousands, or more), service requests for medical imaging devices are frequent, for example, due to failure of MR coils or other field replaceable units (FRU) as electrocardiogram (ECG)-units. The standard procedure for a service request is that clinical staff call the service, for example using a telephone, and explain the problem. If the problem cannot be resolved telephonically, a service person may be dispatched by the vendor of the medical imaging device to perform requisite repair. This can be time consuming; however, depending on the severity of the problem, the medical imaging device may be able to continue to be used while awaiting the servicing, albeit possibly with reduced functionality.

Typically, clinical staff become aware of a problem with a medical imaging device or other reason for a service request when they are currently setting up or executing an imaging workflow. Often, the imaging workflow does not need to be stopped immediately, but can (or must) be continued in some form to ensure patient care is effectively provided. If the current imaging workflow cannot be interrupted, then staff need to remember the problem and call the service later, when they have the time to do this. This delay can cause several problems. For example, staff need to explain the problem verbally from memory, so that they may forget some aspects or details of the event. The service call can be issued with a delay relative to first noticing of the failure which may actually be quite long if such tasks are typically done at the end of a working day due to high patient load. In addition, the staff may need to revisit the imaging suite to recall relevant details of the failure. Transfer of information about the problem with the medical imaging device between work shifts may also be incomplete, leading the next work shift to discover the problem anew. Conversely, if staff decide to stop the imaging workflow immediately to report a device failure or at least call the service within the workflow, then the workflow is delayed, and the patient may become aware of the problem and may be intimidated.

The following discloses certain improvements to overcome these problems and others.

### SUMMARY

In one aspect, a medical imaging system includes a medical imaging device configured to acquire imaging data of an associated imaging subject disposed in an examination region of the medical imaging device; an electronic controller including a display and at least one user input device, the electronic controller configured to implement an imaging workflow graphical user interface (GUI) enabling an associated user to set up an imaging workflow, execute the imaging workflow to acquire imaging data of the associated imaging subject using a medical imaging device, and display at least one medical image of the associated imaging subject generated by processing of the imaging data acquired by the execution of the imaging workflow; a problem reporting GUI dialog hosted on the electronic controller and integrated with the workflow UI, wherein the problem reporting GUI is configured to receive a problem report indicating a problem with the medical imaging device; and a problem remediation tool configured to perform at least one remedial action in response to receiving the problem report via the problem reporting GUI dialog.

In another aspect, a non-transitory storage medium stores instructions readable and executable by an electronic controller of a medical imaging device to perform a problem reporting and remediation method including providing a problem reporting GUI dialog integrated with an imaging workflow GUI implemented by the electronic controller, the problem reporting GUI configured to receive a problem report indicating a problem with the medical imaging device during execution of an imaging workflow performed by the electronic controller to acquire imaging data of an imaging subject using the medical imaging device; and performing at least one remedial action in response to receiving the problem report via the problem reporting GUI dialog.

In another aspect, a medical imaging system includes a customer interface connected via an electronic data network to receive service requests pertaining to medical imaging devices used for execution of imaging workflows from electronic controllers of the medical imaging devices; an electronic processor programmed to perform a service request remediation method including receiving, via the customer interface, a service request pertaining to a medical imaging device being used for execution of an imaging workflow from the electronic controller of the medical imaging device; selecting a service GUI dialog for remediating the service request; configuring the service GUI dialog based on information related to the imaging workflow being executed by the medical imaging device to provide a workflow-specific service GUI dialog, wherein the information is provided by the electronic controller of the medical imaging device; and presenting the workflow-specific service GUI dialog on the electronic controller of the medical imaging device.

One advantage resides in reducing downtime of a medical device due to maintenance.

Another advantage resides in providing a user dialog to allow a user to request maintenance of a medical device.

Another advantage resides in providing a user dialog implemented in a controller of a medical device to allow a user to request maintenance of the medical device.

Another advantage results in fewer errors and delays in reporting service issues for a medical device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative medical imaging system in accordance with the present disclosure.

FIG. 2 diagrammatically illustrates a problem reporting and remediation method using the medical imaging system of FIG. 1.

FIG. 3 diagrammatically illustrates a nonlimiting illustrative example of a display presented by a workflow graphical user interface (GUI) of a magnetic resonance (MR) imaging device showing MR coil configuration, and including nonlimiting illustrative examples of a problem reporting GUI dialog and a service dialog.

### DETAILED DESCRIPTION

When a problem arises with a medical imaging device, it often first manifests during a medical imaging examination being performed using that device. If the problem does not prevent completion of the imaging examination, then the examination will usually be completed, and thereafter a service request may be submitted, e.g., telephonically. This approach has various disadvantages, such as potential for the operator to forget to report the problem, inaccurate reporting of the symptoms and details of the problem due to the time lapse between observation and reporting, and overall delay in initiating the servicing. A further problem is that the operator may be unaware of how to handle the problem, and may for example abort the imaging examination when in fact it could have been continued (possibly with some modifications). Yet a further problem is that subsequent operators may not be made aware of the problem, so that a subsequent operator may also be surprised when the problem manifests in a later examination.

The following provides a reporting tool implemented in and integrated with the electronic controller of the medical imaging device, that permits easy reporting of a problem with the medical imaging device. The controller already has a workflow graphical user interface (GUI) with numerous windows for enabling the user to set up various components of the imaging device, such as selecting whether to use a component, configuring the component for a specific imaging examination, or so forth. The reporting tool is added as a check box labeled “Request service” or a similar problem reporting GUI dialog.

Upon requesting service via the problem reporting GUI dialog dedicated for that purpose, a workflow dependent guided service dialog is executed. This dialog is a pre-designed and potentially branching dialog that provides guidance on whether/how to proceed with the imaging examination based on the received information. In another example, this dialog can dynamically change based on, for example, design input, artificial intelligence (AI) input, and so forth. Advantageously, since the reporting tool is integrated with the electronic controller of the medical imaging device, the workflow dependent guided service dialog has immediate access to real-time information about what imaging examination is being performed and the current state of the imaging examination, along with a time-stamp of machine log data. If some piece of information is not directly extractable from the controller, the dialog may request that information from the operator. With this available knowledge, the workflow dependent guided service dialog can recommend possible tests or repairs (e.g., recommending restarting the malfunctioning component). If the component cannot be immediately repaired, the workflow dependent guided service dialog can recommend workarounds to enable completing the imaging examination to obtain clinically useful images. In one embodiment, the workaround is performed in response to a user confirmation of one or more recommended remedial actions comprising workarounds for completion of the imaging examination that are presented via a service GUI dialog. In another example, the workflow dependent guided service dialog can pass information to the scheduler to determine which of the scheduled examinations can continue, either as planned or with modifications to the workflow.

Furthermore, the real-time information available to the workflow dependent guided service dialog enables it to time any information requests or other interaction with the operator in a manner that minimizes interference with the execution of the imaging workflow of the imaging examination in-progress. For example, the workflow dependent guided service dialog can monitor user inputs to the electronic controller to determine the current state of the imaging workflow, such as whether the operator is currently setting up the next scan, and delay asking for further information until it detects that the next scan is fully set up and started (at which point the operator may be idle for a few minutes as the scan executes, and hence is available to answer questions).

The collected information on the problem, including any test results, along with machine log data generated around the time that the problem manifested, are automatically compiled into a service request that is uploaded to the vendor or other maintenance service provider of the medical imaging device. This minimizes delay in scheduling service (if required). The status of the service request is also sent back to the reporting tool. As the reporting tool advantageously is integrated in the electronic controller of the imaging device, it automatically provides suitable notification to the operator of the problem, and of the current status of the service request, for example by displaying the received status of the transmitted service request as a service request status message integrated with the workflow GUI. This information is also provided to any future operators (e.g., at a shift change), until the service request is fully resolved. The status information may optionally also include recommended workarounds that were generated by the workflow dependent guided service dialog. If the next imaging examination is a different imaging examination, the workflow dependent guided service dialog may adapt the recommended workaround to the next examination (again, advantageously leveraging information about that examination which is advantageously available in real-time due to integration of the reporting tool into the controller of the imaging device).

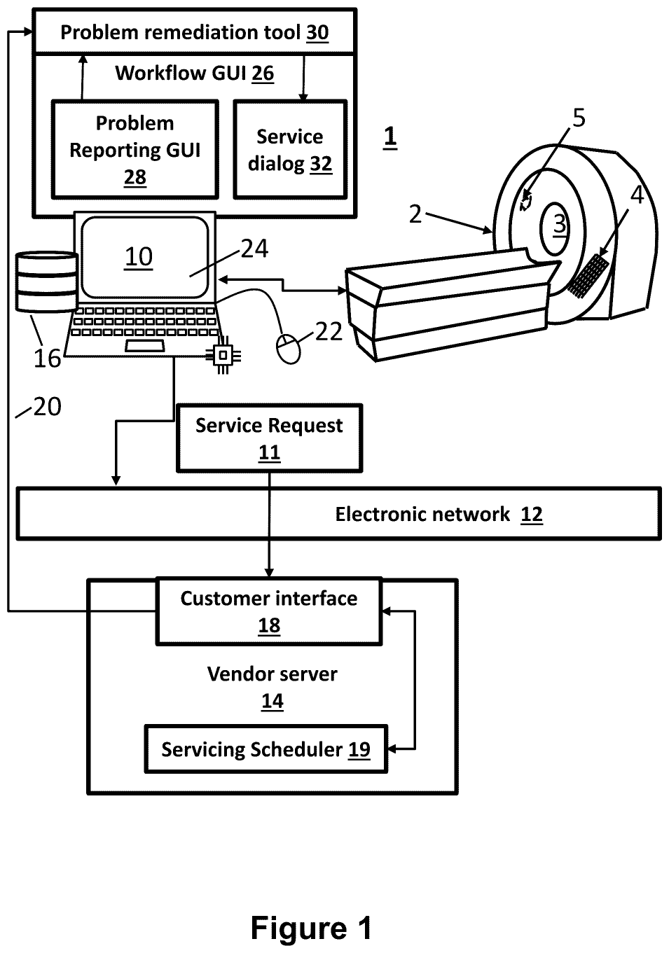

With reference to FIG. 1, an illustrative medical imaging system 1 is diagrammatically shown. The medical imaging system 1 includes a medical imaging device 2 having an examination region 3 for receiving a patient or other imaging subject (e.g., a bore in some medical imaging device designs). The medical imaging device 2 includes a sensor 4 configured to acquire imaging data of an associated imaging subject disposed in the examination region 3 of the medical imaging device 2. As shown in FIG. 1, the medical imaging device 2 includes a computed tomography (CT) imaging device 2 with the sensor 4 comprising an X-ray detector array which detects X-rays emanating from an X-ray tube 5 after passing through the examination region 3 (and hence through the patient loaded therein). By way of some non-limiting illustrative examples, the medical imaging device 2 can be the illustrative CT imaging device 2, or can be a magnetic resonance (MR) imaging device 2 including the sensor 4 comprising one or more MR receive coils, a positron emission tomography (PET) imaging device 2 including the sensor 4 comprising one or more radiation detector rings sensitive to 511 keV radiation, a single photon emission computed tomography (SPECT) imaging device 2 including the sensor 4 comprising a plurality of radiation detectors; an ultrasound imaging device 2 including the sensor 4 comprising an ultrasound transducer array, an interventional radiology (IR) device, or so forth; or may be another type of medical device such as an image guided therapy (IGT) system, a mechanical ventilator, a multifunction patient monitor, a radiation therapy device, or so forth. The medical imaging system 1 is computerized, and may be connected to an electronic network.

The medical imaging device 2 can include (or, viewed alternatively, be operatively connected with) an electronic controller 10, which is configured to implement an imaging workflow graphical user interface (GUI) 26 enabling an operator (or, more generally, a user) to set up an imaging workflow, execute the imaging workflow to acquire imaging data of a patient (or other imaging subject) using the medical imaging device 2, and display at least one medical image of the patient generated by processing of the imaging data acquired by the execution of the imaging workflow. The electronic controller 10 is connected with an electronic data network 12 (e.g., a hospital IT network, radiology department IT network, or the like). The electronic data network 12 may optionally also include the Internet, e.g., the electronic controller 10 may connect with a remote server such as a server 14 of the medical imaging device vendor via the Internet accessed through a firewall of the hospital IT network as a nonlimiting illustrative example. It will be appreciated that the electronic network 12 typically has many other medical devices (possibly including other medical imaging devices), and potentially other types of devices, connected therewith, For example, the electronic network 12 may be a hospital network that interconnects personal computers used by hospital personnel, various types of medical devices including the illustrative medical device 10, cellular telephones or other mobile devices of hospital personnel, and/or so forth.

As further shown in FIG. 1, the vendor server 14 is hosted by the vendor of the medical imaging device 2. The vendor server 14 may, for example, be a single server computer, or a cluster of servers, or a cloud-based ad hoc server network, or so forth, and is accessible via the Internet.

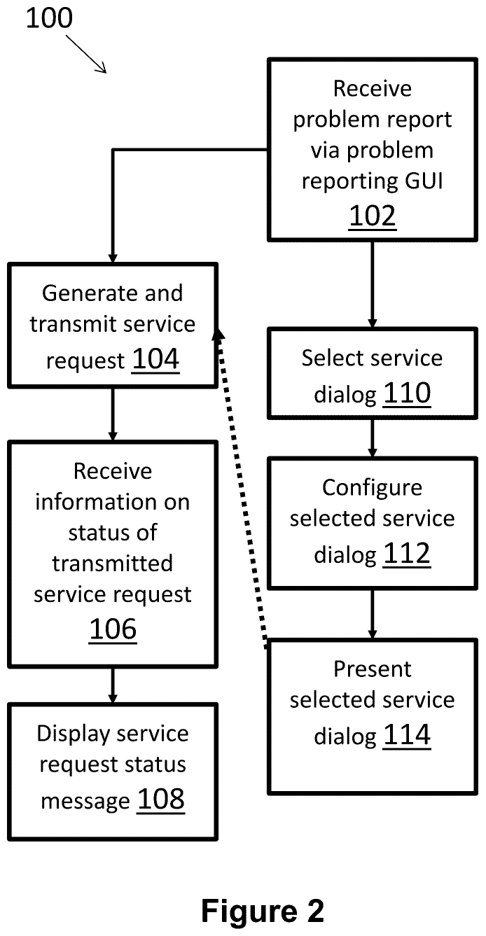

The electronic controller 10 of the medical imaging device 2 includes or is operatively connected with a non-transitory storage medium 16 storing instructions readable and executable by the electronic processor 14 to implement a problem remediation tool 30 which performs a problem reporting and remediation method or process 100 (see example shown in FIG. 2). Note that the medical imaging device 2 may comprise multiple components: for example, a magnetic resonance (MR) imaging device may include the MR scanner (including the main magnet, gradient coils, radio frequency coils, MR receive coils, ECG detection unit, respiratory belt, and so forth) located in the magnet room and an MR scanner controller comprising the electronic processor 14 which is located in a separate, adjacent control room. In this example, the medical imaging device 2 is considered to include both the MR scanner and the MR scanner controller. As another example, the medical imaging device 2 could be an image guided therapy (IGT) system including a medical imaging device and hardware for performing an intravascular procedure or other medical procedure under guidance of the medical imaging device.

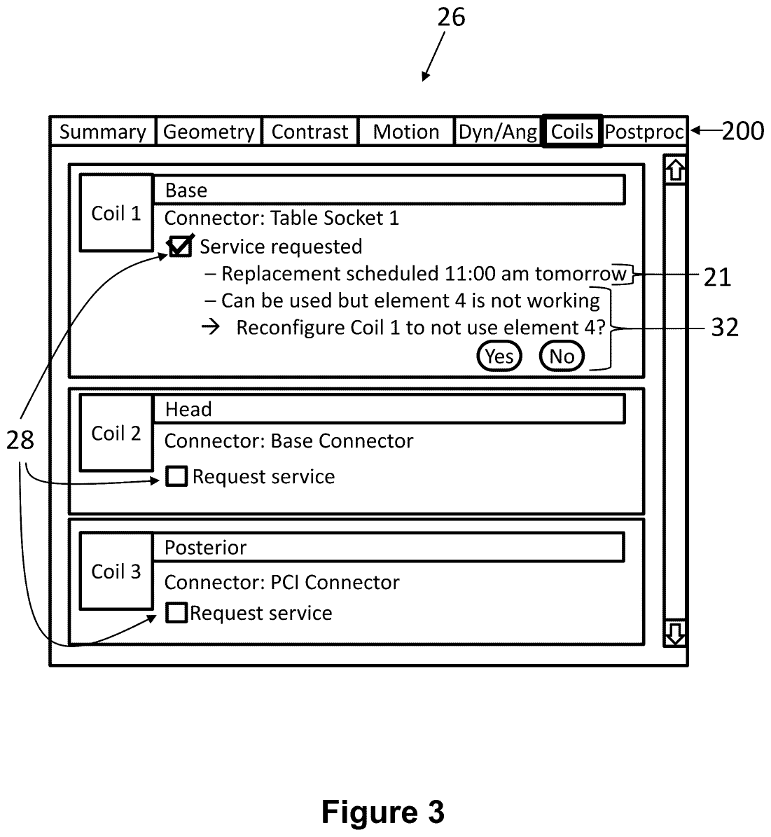

The problem remediation tool 30 running on the electronic controller 10 of the medical imaging device 2 is configured to send a service request 11 pertaining to execution of an imaging workflow being performed using the medical imaging device 2 from electronic controller 10 to a customer interface 18 of the vender server 14. The customer interface 18 may, for example, be implemented as an application programming interface (API), a web portal, or so forth. The service request 11 may, by way of nonlimiting illustrative example, pertain to a problem with the medical imaging device 2 (where, as used herein, the medical imaging device 2 may include an interoperative component such as a contrast injector operative to administer an intravascular contrast agent to the imaging subject during the execution of the imaging workflow, an MR coil provided by a third party to be used with an MR imaging device, or so forth). A servicing scheduler 19 implemented on the vendor server 14 suitably schedules a service call (where a field service engineer goes to the hospital to resolve the problem) or other servicing to resolve the service request 11. Information on status of the transmitted service request 11 is received from the customer interface 18, and the received status of the transmitted service request 11 is displayed as a service request status message 21 integrated with the workflow GUI 26 (see, e.g., example service request status message 21 shown in the nonlimiting illustrative example of FIG. 3).

As shown in FIG. 1, the service request 11 is transferred or uploaded to a customer interface 18 of the vendor server 14 of the vendor via the electronic network 12 (which, as previously noted, may include the Internet). The service request 11 may be received at the customer interface 18 via the Internet (in embodiments in which the customer interface 18 comprises a website or API) or may be a text message service request or the like.

The medical device electronic controller 10 includes at least one user input device 22 (e.g., a mouse, a keyboard, a trackball, and/or the like) 22, and a display device 24 (e.g., an LCD display, plasma display, and/or so forth). The electronic controller 10 is configured to implement the imaging workflow graphical user interface (GUI) 26 on the display device 24 that enables an the operator (or, more generally, user) of the medical imaging system 1 to set up an imaging workflow, execute the imaging workflow to acquire imaging data of the patient (or, more generally, imaging subject), and display at least one medical image of the patient generated by processing of the imaging data acquired by the execution of the imaging workflow on the display device 24.

As disclosed herein, the electronic controller 10 of the medical imaging device 2 is further configured to provide the reporting tool 30. To this end, the electronic controller 10 is configured to additionally implement a problem reporting GUI dialog 28 hosted on the electronic controller 10 and integrated with the workflow GUI 26. For example, the problem reporting GUI dialog 28 may comprise a problem reporting selection input (e.g., a checkbox) displayed by the imaging workflow GUI 26 (see example in FIG. 3). The problem reporting GUI dialog 28 is configured to receive a problem report indicating a problem with the imaging device being used for the execution of the imaging workflow. In some examples, the problem report indicates the problem with the execution of the imaging workflow is one of a problem with the medical imaging device 2 or an interoperating component such as a contrast injector operative to administer an intravascular contrast agent to the associated imaging subject disposed in the examination region 3 during the execution of the imaging workflow, an MR coil provided by a third party to be used with an MR imaging device, or so forth.

The problem remediation tool 30 implemented in the electronic controller 10 is further configured to additionally perform at least one remedial action in response to receiving the problem report via the problem reporting GUI dialog 28. In one example embodiment, the at least one remedial action performed by the problem remediation tool 30 includes generating the service request 11 for the problem report, and transmitting the service request 11 via the electronic data network 12 to the customer interface 18 of the electronic processor 14 of the vendor.

In some embodiments, the at least one remedial action performed by the problem remediation tool 30 additionally or alternatively includes selecting a service dialog 32 for remediating the problem. The service dialog 32 is, in some embodiments, configured based on a state of the imaging workflow at a time of receipt of the problem report. The service dialog 32 is presented on the display device 24 via the electronic controller 10. In one embodiment, the workaround is performed in response to a user confirmation of one or more recommended remedial actions comprising workarounds for completion of the imaging examination that are presented via the service GUI dialog 32. As shown in the nonlimiting illustrative example of FIG. 3, the service dialog 32 is presented in a window of the imaging workflow GUI 26.

With reference to FIG. 2, and with continuing reference to FIG. 1, an illustrative embodiment of an instance of the problem reporting and remediation method 100 suitably performed by the problem remediation tool 30 of FIG. 1 is diagrammatically shown as a flowchart.

At an operation 102, a problem report is received via the problem reporting GUI 28 indicating a problem with the medical imaging device 2 being used in the execution of the imaging workflow. For example, this may entail the user checking a checkbox next to (or otherwise associated with) the component experiencing the problem.

In one remedial path, in an operation 104 a service request 11 is generated for the problem report received at operation 102, and the service request 11 is transmitted via the electronic data network 12 to the customer interface 18 via which service requests pertaining to medical devices are received. The servicing scheduler 19 (see FIG. 1) schedules servicing, and in an operation 106 information 20 (see FIG. 1) on the status of the transmitted service request is received from the customer interface 18. In an operation 108, the received status of the transmitted service request 11 is displayed as a service request status message 21 (see FIG. 3) integrated with the workflow GUI 26.

In another remedial path, at an operation 110, a service dialog 32 is selected for remediating the reported problem received at the operation 102. For example, the electronic controller 10 may store service dialogs for various components and/or common problems of the medical imaging device 2, and the selection is then based on the component experiencing the problem as indicated by the user's interaction with the problem reporting GUI 28. Each stored service dialog may, for example, be represented as a tree structure or graph with each node representing an action and paths between the nodes indicating a sequence of actions represented by the connected nodes. The paths may be branching, with a decision on which branch to follow being made based on information received from the user (e.g., test results) or based on information on the state of the imaging workflow which is advantageously directly obtainable from the hosting electronic controller 10. In some embodiments, the service dialog 32 can be selected, based on machine log data generated by the medical imaging device 2 by an AI component implemented in the problem remediation tool 30. The AI component implemented in the problem remediation tool 30 is configured to predict a set of workaround options (optionally with likelihoods of success) that are based on, for example, e.g., current machine log data generated by the medical imaging device 2, and determined based on historical workaround solutions.

At an optional operation 112, the selected service dialog may be configured based on a state of the imaging workflow at a time of receipt of the problem report. For example, if the current state of the imaging workflow is one in which the operator is busy, the configuration may include delaying presenting advice for remediating the problem until a point in the workflow is reached at which the operator is idle. As another example, the recommended workaround for the problem may depend on the type of imaging workflow currently being executed. As the problem remediation tool 30 is hosted at the electronic controller 10 of the medical imaging device 2, the state of the imaging workflow is advantageously directly obtainable from the controller 10.

At an operation 114, the selected (and optionally configured) service dialog is presented via the electronic controller 10, for example in the imaging workflow GUI 26 (e.g., as a pop-up window of the imaging workflow GUI 26, or in a dedicated field of the imaging workflow GUI 26, or so forth. As noted, the presented service dialog 32 may optionally include receiving information from the user (e.g., test results) and selecting a branch of the tree or graph determining which recommendation is presented next, so that the presentation of the service dialog is updated over time. In another example, the selected workflow-specific service dialog 26 may include a dialog or dialog portion (i.e., the problem reporting GUI dialog 28) recommending one or more remedial actions comprising workarounds for completion of the imaging examination, which can be ordered based on a state of the imaging workflow determined from the information provided by the electronic controller 10. A delay can be inserted in the workflow-specific service dialog 26 based on a state of the imaging workflow determined from the information provided by the electronic controller 10.

It will be appreciated that the flowchart shown in FIG. 2 is nonlimiting. For example, the selected service dialog presented in the operation 114 may include guidance to resolve the problem, and the results of this guidance can be included in the service request generated at the operation 104. This is diagrammatically shown in FIG. 2 with a dashed arrow. Other correlations between the operations 104, 106, and 108, and the operations 110, 112, and 114 are also contemplated.

With reference now to FIG. 3, a nonlimiting illustrative example of operation of the problem remediation tool 30 is shown for an example of an MR imaging device. As shown in FIG. 3, the workflow GUI 26 in this example has a number of user-selectable tabs 200, and currently the “Coils” tab is selected, thus bringing up a list of MR coils of the MR imaging device, including a “Coil 1” which is a base coil, a “Coil 2” which is a head coil, and a “Coil 3” which is a posterior coil. The problem reporting GUI dialog 28 in this nonlimiting illustrative example includes a checkbox associated with each of “Coil 1”, “Coil 2”, and “Coil 3”. For “Coil 2” and “Coil 3”, this checkbox is labeled “Request service”. For “Coil 1” the user has selected the corresponding checkbox (thus indicating a problem with that coil) and so the checkbox for “Coil 1” is labeled “Service requested”. As further shown in FIG. 3, this has resulted in the operations 104, 106, and 108 of FIG. 2 being performed to generate and transmit a service request for “Coil 1” to the vendor and to receive back the information on the status of this service request which is displayed per operation 108 in FIG. 3 as the message 21: “Replacement scheduled 11:00 am tomorrow”. Furthermore, the selection of the checkbox for “Coil 1” has also resulted in the operations 110, 112, and 114 of FIG. 2 being executed to present a selected service dialog 32, which in this case states: “Can be used but element 4 is not working” and “Reposition Coil 1 to not use element 4?” along with user-selectable “Yes” or “No” buttons. In this example, if the user selected “Yes” then the workflow GUI will be automatically updated to account for not using element 4 of “Coil 1”, and thereby to continue the workflow.

While FIG. 3 shows an example for a problem with an MR coil, it will be appreciated that a similar approach can be used for other components. For example, the tabs 200 shown in FIG. 3 include a “Motion” or “Dynamics” tab, which when selected may suitably bring up a display showing information about when to trigger a contrast injector for intravascular administration of a magnetic imaging contrast agent to the patient using a contrast injector such that this injection is done in synchronization with the imaging workflow. In this case, the display of the contrast injector information can include a similar problem reporting GUI dialog 28 with a checkbox the user can check to indicate a problem with the contrast injector (or with a component thereof, such as the contrast agent supply). As yet another example, the “Postproc” tab may bring up information on image postprocessing tools (e.g., image filters) and the user can use the problem reporting GUI dialog 28 comprising a checkbox next to each postprocessing tool to indicate a problem with that postprocessing tool. While the foregoing examples are for an MR imaging device, a similar approach can be employed for medical imaging devices of other modalities, e.g., a CT imaging device may include a tab for configuring the X-ray tube and associated problem reporting GUI dialog checkbox for reporting a problem with the X-ray tube.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A medical imaging system, comprising:
a medical imaging device configured to acquire imaging data of an associated imaging subject disposed in an examination region of the medical imaging device;
an electronic controller including a display and at least one user input device, the electronic controller configured to implement an imaging workflow graphical user interface (GUI) enabling an associated user to set up an imaging workflow, execute the imaging workflow to acquire imaging data of the associated imaging subject using the medical imaging device, and display at least one medical image of the associated imaging subject generated by processing of the imaging data acquired by the execution of the imaging workflow;
a problem reporting GUI dialog hosted on the electronic controller and integrated with the workflow UI, wherein the problem reporting GUI is configured to receive a problem report indicating a problem with the medical imaging device; and
a problem remediation tool configured to perform at least one remedial action in response to receiving the problem report via the problem reporting GUI dialog.

2. The medical imaging system of claim 1, wherein the electronic controller is operatively connected with an electronic data network, and the at least one remedial action performed by the problem remediation tool includes:
generating a service request for the problem report; and
transmitting the service request via the electronic data network to a customer interface via which service requests pertaining to medical devices are received;
receiving information on status of the transmitted service request from the customer interface; and
displaying the received status of the transmitted service request as a service request status message integrated with the workflow GUI.

3. The medical imaging system of claim 1, wherein the at least one remedial action performed by the problem remediation tool includes:
selecting a service dialog for remediating the problem; and
presenting the service dialog via the electronic controller.

4. The medical imaging system of claim 3, wherein the at least one remedial action further includes:
prior to presenting the service dialog, configuring the service dialog based on a state of the imaging workflow at a time of receipt of the problem report.

5. The medical imaging system of claim 4, wherein the problem remediation tool is hosted on the electronic controller and presents the service dialog via the imaging workflow GUI.

6. The medical imaging system of claim 1, wherein the problem reporting GUI dialog comprises a problem reporting selection input displayed by the imaging workflow GUI.

7. The medical imaging system of claim 1, wherein the medical imaging device comprises at least one of:
a computed tomography (CT) imaging device;
a magnetic resonance (MR) imaging device;
a positron emission tomography (PET) imaging device;
a single photon emission computed tomography (SPECT) imaging device;
an interventional radiology (IR) imaging device;
an image guided therapy (IGT) imaging device; and/or
an ultrasound imaging device.

8. A non-transitory storage medium storing instructions readable and executable by an electronic controller of a medical imaging device to perform a problem reporting and remediation method comprising:
providing a problem reporting graphical user interface (GUI) dialog integrated with an imaging workflow GUI implemented by the electronic controller, the problem reporting GUI configured to receive a problem report indicating a problem with the medical imaging device during execution of an imaging workflow performed by the electronic controller to acquire imaging data of an imaging subject using the medical imaging device; and
performing at least one remedial action in response to receiving the problem report via the problem reporting GUI dialog.

9. The non-transitory storage medium of claim 8, wherein the electronic controller is operatively connected with an electronic data network, and the performing of the at least one remedial action includes:
generating a service request for the problem report;
transmitting the service request via the electronic data network to a customer interface via which service requests pertaining to medical devices are received;
receiving information on status of the transmitted service request from the customer interface; and
displaying the received status of the transmitted service request as a service request status message integrated with the workflow GUI.

10. The non-transitory storage medium of claim 8, wherein the performing of the at least one remedial action includes:
selecting a service dialog for remediating the problem report; and
presenting the service dialog on the electronic controller.

11. The non-transitory storage medium of claim 10, wherein the performing of the at least one remedial action further includes:
prior to presenting the service dialog, configuring the service dialog based on a state of the imaging workflow at a time of receipt of the problem report.

12. The non-transitory storage medium of claim 10, wherein the service dialog is presented via the imaging workflow GUI.

13. The non-transitory storage medium of claim 8, wherein the problem reporting GUI dialog comprises a problem reporting selection input displayed by the imaging workflow GUI.

14. A medical imaging system, comprising:
a customer interface connected via an electronic data network to receive service requests pertaining to medical imaging devices used for execution of imaging workflows from electronic controllers of the medical imaging devices;
an electronic processor programmed to perform a service request remediation method including:
receiving, via the customer interface, a service request pertaining to a medical imaging device being used for execution of an imaging workflow from the electronic controller of the medical imaging device;
selecting a service GUI dialog for remediating the service request;
configuring the service GUI dialog based on information related to the imaging workflow being executed by the medical imaging device to provide a workflow-specific service GUI dialog, wherein the information is provided by the electronic controller of the medical imaging device; and
presenting the workflow-specific service GUI dialog on the electronic controller of the medical imaging device.

15. The medical imaging system of claim 14, wherein the workflow-specific service GUI dialog includes:
a dialog or dialog portion recommending one or more remedial actions comprising workarounds for completion of the imaging examination.

16. The medical imaging system of claim 14, wherein the workflow-specific service GUI dialog includes:
a dialog or dialog portion recommending a plurality of remedial actions for the received service request ordered based on a state of the imaging workflow determined from the information provided by the electronic controller.

17. The medical imaging system of claim 16, wherein the workflow-specific service GUI dialog includes:
inserting a delay in the workflow-specific service GUI dialog based on a state of the imaging workflow determined from the information provided by the electronic controller.

18. The medical imaging system of claim 14, wherein the method further includes:
uploading the service request to a server operable by a vendor of the medical device.

19. The medical imaging system of claim 14, wherein the method further includes:
updating the workflow-specific service GUI dialog based on a state of a subsequent imaging workflow to be performed using the medical imaging device.

20. The medical imaging system of claim 14, wherein the electronic processor is integrated with an electronic controller of the medical device.
